(19) United States
(12) Patent Application Publication
(10) Pub. No.: US 2004/0096948 A1
(43) Pub. Date: May 20, 2004

(54) Glycoprotein cleavage protocol for oligosaccharide analysis

(76) Inventors: Yunping Huang, Urbana, IL (US); Yehia S. Mechref, Bloomington, IN (US); Milos V. Novotny, Bloomington, IN (US)

Correspondence Address: BARNES & THORNBURG, 11 SOUTH MERIDIAN, INDIANAPOLIS, IN, 46204

(21) Appl. No.: 10/643,502

(22) Filed: Aug. 19, 2003

Related U.S. Application Data
(60) Provisional application No. 60/426,921, filed on Nov. 15, 2002.

(51) Int. Cl. C12P 19/04; C08B 37/00

(52) U.S. Cl. 435/101; 536/123; 536/18.7

(57) ABSTRACT

An ammonium-based β-elimination cleavage of linked oligosaccharides from glycoproteins is described. The method enables the isolation of glycoprotein-derived oligosaccharides having a reducing end which enables subsequent derivatization for chromatographic and/or mass spectral analysis. The described glycoprotein cleavage protocol enables structural investigations using low microgram quantities of glycoproteins.

This application claims priority under 35 U.S.C. § 119(e) to U.S. Provisional Application Serial No. 60/426,921, filed on Nov. 15, 2002, the disclosure of which is hereby incorporated by reference herein.

### GOVERNMENT RIGHTS

Research relating to this invention was supported in part by the U.S. Government under Grant No. GM24349 awarded from the National Institute of Health. The U.S. Government may have certain rights in this invention.

### FIELD OF INVENTION

This invention relates to glycoprotein analysis. More particularly, this invention is directed to a method for non-reductive degradation of glycoproteins with release of oligosaccharides for derivation and/or analysis.

### BACKGROUND AND SUMMARY OF THE INVENTION

The roles of glycosylated proteins, both known and suspected, have fascinated scientists for more than two decades. The compelling evidence for participation of glycosylated structures in a multitude of cellular processes has focused research efforts on detailed structural investigations of glycosylated proteins and methodological developments to implement such investigations.

From the analytical viewpoint, the contemporary glycobiology presents particular challenges in terms of needed sensitivity and structural complexity. Since the detailed structures of glycans cannot be currently assessed at the level of intact glycoproteins, the first step in glycoprotein oligosaccharide analysis involves their release from the polypeptide backbone. While the sensitivity potential for the final glycan measurements has been enhanced through the recent developments in matrix-assisted laser desorption/ionization (MALDI) mass spectroscopy, electrospray mass spectrometry (MS), and laser-induced fluorescence (LIF) detection, the preliminary steps for use of such developments, such as cleavage (enzymatic or chemical), microscale isolation, and separation can all be a bottleneck for the overall analytical scheme.

Extensive structural identifications has often necessitated amounts of initial glycoprotein samples on the order of milligrams to grams. The methodological advances of the last several years have made significant inroads into the structural analysis of asparagine-linked (N-linked) oligosaccharides that can now often be assessed with very small sample quantities. This situation has been facilitated by the availability of peptide N-glycosidases and endoglycosidases that readily release N-glycans. In contrast, the release and recovery of serine- and threonine-linked (O-linked) glycans have remained a very challenging problem due to the limited availability and specificity of O-glycanases that would be suitable for this task. This situation makes chemical cleavage methods the only viable alternative for the analysis of O-linked oligosaccharides in glycoproteins at present.

Alkaline β-elimination and hydrazinolysis are the two most commonly used chemical cleavage methods for glycan release. They both suffer from several problems. Alkaline β-elimination is viewed as the most reliable and universal method in existence for the release of O-linked oligosaccharides. However, the presence of a strong reducing agent, which converts glycans to their respective alditols, is necessary to minimize the peeling reactions caused by the alkaline medium. Conversion to alditols prevents the reductive amination needed for the attachment of a fluorophore or of a polyvalent coupling to a lipid or protein for a subsequent immunoassay. Alkaline β-elimination, embodied in the widely used Carlson procedure, is also difficult to practice at microscale, as the minute quantities of released glycans are overwhelmed by excessive amounts of salts.

While hydrazinolysis is the most widely used approach to yield reducing glycans, it constitutes a tedious procedure with many needed precautions. Additionally, it leads to chemical modification of the original glycans such as the loss of N-acetyl and N-glycolyl groups from the amino sugar residues. O-Acyl substitutions in sialic acids are not also retained upon hydrazinolysis. More recently, improvements were sought, through the use of 70% (w/v) aqueous ethylamine to release nonreductively the O-linked oligosaccharides from glycoproteins. Unfortunately, the overall reaction yields were low, as the oligosaccharides underwent significant peeling reaction or other forms of degradation.

Whereas β-elimination is generally effective, the formation of alditols makes it difficult to investigate released glycans in complex mixtures. The lack of a chromophore in these structures during liquid-phase separations makes their detection difficult. UV absorbance measurements below 200 nm are relatively insensitive, as is MS detection due to the low ionization efficiency with intact glycans. In contrast, the cleavage procedures yielding the reducing end make it relatively easy to attach a chromophore or a fluorophore through reductive amination with aromatic amines prior to a chromatographic or electrophoretic separation. Derivatization featuring appropriate structural moieties can also be beneficial in enhancing the sensitivity of MS investigations.

The enormous significance of O-linked glycans in cellular recognition, tissue-specific regulation, and disease necessitates the development of more sensitive structural methodologies. These necessarily include more effective means of glycan cleavage and recovery. The present invention provides an effective procedure for glycan cleavage that can be conducted on the microscale level. It enables a glycan-releasing non-reducing hydrolysis characterized by mild reaction conditions (without peeling reactions), the recovery of oligosaccharides having a reducing end, and by easy removal of the excess reagents. The present method is an ammonia-based β-elimination, which provides intact, reducing glycans with high yield enabling structure elucidation studies with small (<1 mg) sample sizes. The advantages of recovering oligosaccharides with a reducing end are demonstrated with the use of reductive amination and other coupling chemistries to covalently attach spectroscopically desirable moieties for MALDI-MS and capillary electrophoresis with LIF detection.

### BRIEF DESCRIPTION OF THE DRAWINGS

FIG. 1 depicts a reaction scheme for cleavage of O-linked or N-linked oligosaccharides from glycoproteins in accordance with present invention.

FIG. 2 is a matrix-assisted laser desorption/ionization (MALDI) mass spectra of (a) original maltoheptose and (b) maltoheptose subjected to ammonia-based β-elimination.

FIG. 3 is a negative ion MALDI mass spectra of the glycans cleaved from bovine fetuin by ammonia-based β-elimination. Matrix peaks are labeled with asterisks, and peaks originating from a loss of carboxylic acid groups are marked with small, filled circles. Symbols, filled in diamond (♦), GalNAc; filled in square (▪), GlcNAc; open square (□), Gal; open circle (∘), Man; filled in triangle (▴), NeuAc.

FIG. 4 are MALDI mass spectra of the glycans cleaved from a 10-μg sample of the human milk bile salt-simulated lipase: (a) positive-ion mode and (b) negative-ion mode.

FIG. 5 is a negative ion MALDI mass spectrum of 2-aminobenzamide-labeled glycans cleaved from bovine fetulin. Symbols: filled in star (★), 2-aminobenzamide tag; other symbols as in FIG. 3.

FIG. 6 are electropherograms of APTS-labeled glycans: (a) dextran ladder (DP, degree of polymerization); N- and O-glycans cleaved from (b) fetuin, (c) asialofetuin, (d) mucin, and (e) BSSL. Conditions: buffer, 25 mM Tris-HCl (pH 6.5); voltage-20 kV; current: 13 μA; injection, 5 s (hydrodynamically at 15-cm height difference).

### DETAILED DESCRIPTION OF THE INVENTION

The present invention is based on a β-elimination type procedure for release of linked oligosaccharides from glycoproteins. The procedure provides oligosaccharide products with the reducing end intact. The cleavage conditions effect conversion of the liberated oligosaccharide to glycosylamines-glycosylamine carbonates in an aqueous ammonia/ammonium carbonate medium. Following separation of the reaction products from the aqueous ammonia/ammonium carbonate medium by an iterative solution evaporation protocol. The product glycosylamines are converted quantitatively to the corresponding reducing oligosaccharides by their dissolution in aqueous boric acid. The boric acid reagent is separated from the resulting product mixture by evaporation followed by repeated addition of methanol and evaporation of the resulting solution until the product mixture is free of boric acid. The reducing oligosaccharide products can be separated from the glycosylated protein by-product by dissolving the oligosaccharide mixture in water and separating the solution from the typically insoluble protein by-products. The resulting mixture of glycoprotein derived oligosaccharide, each having a reducing end, can subsequently be derivatized with fluorophoric reagents for capillary electrophoresis or alternatively analyzed with or without derivatization using MALDI mass spectrometry. The nature of the chemistry and processing of the reaction products permits investigation of structurally linked oligosaccharides using very small (even microgram quantities) glycoprotein samples.

Thus in accordance with the present invention there is provided a method for preparing stable oligosaccharide from a glycoprotein having linked oligosaccharides. The method comprises the steps of contacting the glycoprotein with an aqueous solution of ammonium hydroxide and ammonium carbonate for a period of time sufficient to cleave the linked oligosaccharides from the glycoprotein to form oligosaccharide products and deglycosylated protein by-products. In one embodiment the aqueous ammonium hydroxide solution is saturated with ammonium carbonate at room temperature and reaction is carried out by incubation of the reaction mixture in the presence of solid ammonium carbonate. The time required to complete the reaction is dependent on the reaction temperature and ammonium hydroxide concentration in the reaction mixture which parameters can be varied over a wide range of values within the scope of the present invention. Typically when the concentration of ammonium hydroxide is about 20 to about 30%, the deglycosylation reaction is substantially complete after 40 hours at about 60° C. Such times, temperatures and reagent concentrations are not critical to performance of the deglycosylation reaction and such parameters can be varied widely without detracting significantly from the overall efficacy of the ammonia-based P-elimination deglycosylation procedure.

The intermediate glycosylamine products of the deglycosylation reaction can be separated from the excess ammonia hydroxide and the ammonia carbonate reagents by repeated evaporation of water added to the mixture until no salts are detectable in the reaction product mixture.

In so-called microscale applications of the present invention, for example, where the total volume of the reaction mixture is less than about 100 μL of liquid, the reaction mixture can be incubated in the bottom a general purpose microcentrifuge tube with a flat-top snap cap. The adhesive forces (surface tension) between the liquid sample and the walls of the microcentrifuge tube, which are typically made of polypropylene, are strong enough to hold the liquid sample in a position in the conical bottom portion of the tube even when the tube is held in an upside-down position during incubation. A floating device, for example, a sponge having several holes for receiving the tubes, can be used to hold the tubes in the upside-down orientation during incubation in a water bath so that when the tubes are heated from the bottom (the flat top snap cap when the tube is in an upside-down position) the sample can be incubated without evaporative loss from the small liquid sample. This system allows incubation of a 0.5 μL reaction volume at 37° for 24 hours without detachable loss of volume of the reaction mixture.

The intermediate glycosylamines carbonates are converted quantitatively to the corresponding oligosaccharides having a reducing end by their dissolution in a boric acid solution; typically the mixture is held at room temperature or higher for about 30 minutes. The reaction mixture is then dried, and the boric acid reagent is removed from the resulting oligosaccharide products by evaporation under a stream of nitrogen with several additions of methanol to provide a product mixture including the oligosaccharide products having a reducing end and the deglycosylated protein by-products. Separation of the oligosaccharide products from the protein byproducts is accomplished by dissolving the mixture in a small volume of water and centrifuging. The supernatant containing the water soluble oligosaccharide products is easily separated from the insoluble protein by-product. The product mixture in the supernatant can be used directly for a MALDI-MS measurements or the mixture can be optionally dried and the oligosaccharide reaction products collected and optionally derivatized to facilitate subsequent chromatographic separation and analysis.

Ammonium hydroxide has been used to remove the glycan chains from glycopeptides. However, that treatment could not prevent the oligosaccharide peeling reactions in the absence of a reducing agent.

More than a decade ago, nonanalytical researchers were successful in converting the reducing oligosaccharides into the corresponding primary glycosylamines in good (>95%) yields by treatment with saturated aqueous ammonium bicarbonate. Glycosylamine exists in equilibrium with N-glycosylamine carbonate in the reaction solution when ammonium bicarbonate is used. The reaction yield with ammonium bicarbonate was much higher than when concentrated aqueous solutions of ammonium acetate, ammonium formate, or ammonium chloride were used. Evaporation of the excess ammonium bicarbonate results in a complete conversion of N-glycosyl carbonate to glycosylamine, which is very stable in a wide pH range (8.0-10.0). The glycosylamine is converted to a reducing oligosaccharide by lowering the pH of the reaction mixture to about 5 pH.

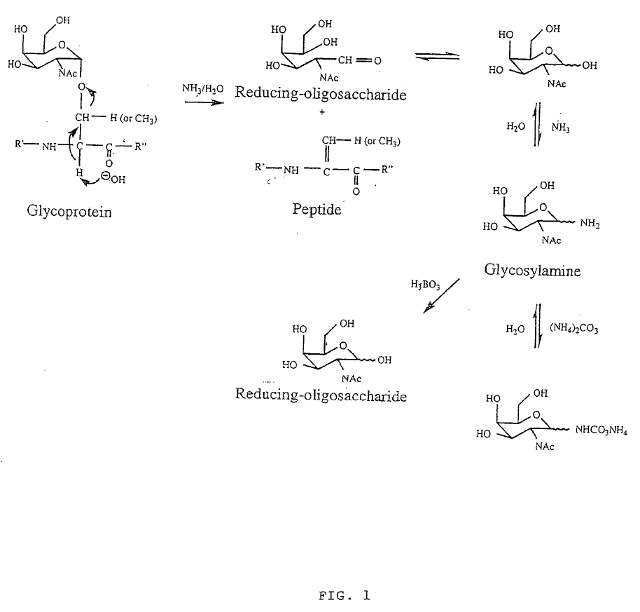

In the present ammonia-based β-elimination protocol (see FIG. 1 where the N-acetylgalactosamine group is depicted as representative of the oligosaccharides cleaved from glycoproteins in accordance with the present invention); ammonium carbonate is utilized to provide the higher alkaline conditions favoring initiation of the elimination reaction. Aqueous ammonium hydroxide solution, instead of the normally used sodium hydroxide solution, was employed to maintain the alkaline conditions (pH 9.5-10.0) necessary for β-elimination to take place. The combination of aqueous ammonium hydroxide (for β-elimination) and ammonium carbonate for converting the cleaved reducing oligosaccharides to glycosylamines has proven to be a most practical reagent combination since both are volatile and easily removable by evaporation, thus eliminating the need for an elaborate desalting step. The addition of boric acid to convert the glycosylamines to the reducing glycans further simplifies the present procedure since excess borate can be easily removed by methanol evaporation. As detailed below, this chemical cleavage procedure was successfully applied to the release of O-glycans from several glycoproteins, such as fetuin, asialofetuin, and mucin, that have been studied extensively in the past. Application of the present method to BSSL at microscale, as seen below, further underscores the analytical potential of the present ammonia-based β-elimination protocol.

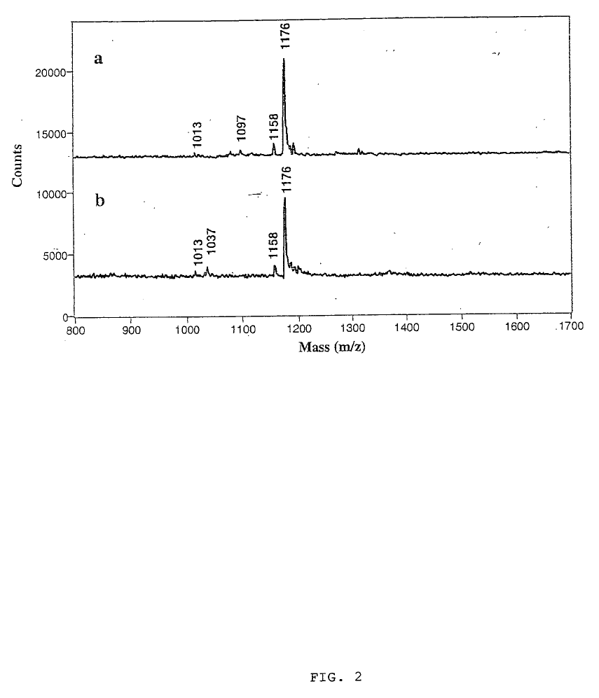

Stability of Reducing Oligosaccharides under the Environment of Ammonia-Based β-elimination. The stability of oligosaccharides under the reaction environment of the present invention was tested using maltoheptaose. FIG. 2a illustrates the mass spectrum of the original maltoheptaose (m/z 1175), while FIG. 2b is recording the maltoheptaose that endured the ammonia-based β-elimination protocol. MALDI mass spectrum of the original maltoheptaose reveals the presence of maltohexaose as an impurity (1023 m/z value in FIG. 2a); this signal does not increase upon treatment of the sample with the reaction mixture (FIG. 2b). This suggests the absence of a significant peeling reaction. The origin of a new, minor signal observed in FIG. 2b at 1037 m/z values is not understood. Even if it were a result of maltoheptaose degradation, the signal is minimal. In addition, when maltoheptaose was treated with aqueous ammonium hydroxide alone (in the absence of ammonium carbonate), it suffered a major peeling reaction as observed in its MALDI mass spectrum (data not shown), suggesting the protective function of ammonium carbonate.

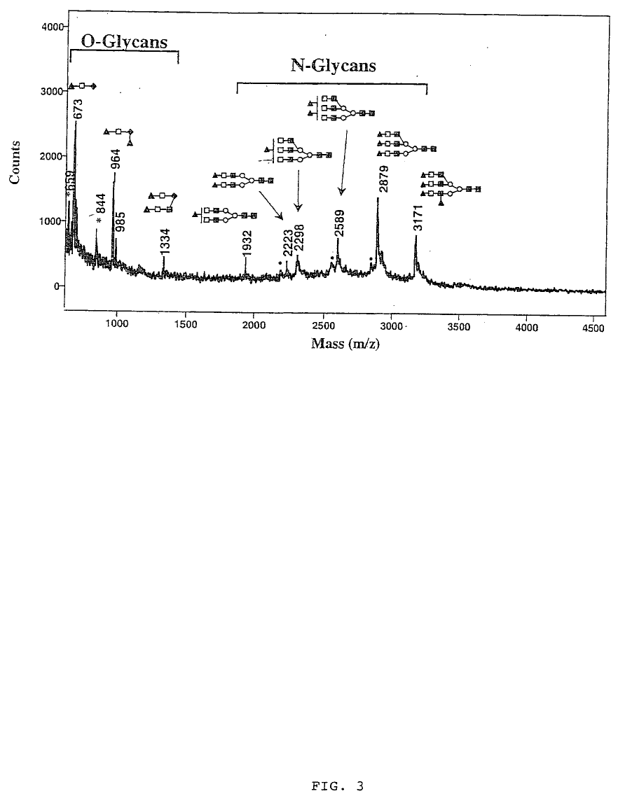

Glycans Released from Fetuin. The procedure was then applied to bovine fetuin, a glycoprotein that possesses both N and O-glycans which have been well-characterized. A negative-ion MALDI mass spectrum of N- and O-glycans released from fetuin is illustrated in FIG. 3. All known N- and O-glycans can be observed in this spectrum, thus illustrating the effectiveness of the described procedure. The relative intensities of the N-glycan signals are exactly the same as those observed in an earlier work using N-glycanases. This suggests a very efficient and quantitative cleavage. No loss of acetyl groups, as often seen in hydrazinolysis, seems to occur. All major m/z values of the mass spectrum observed in FIG. 3 pertain to the intact, original glycan structures. Two peaks labeled with asterisks in FIG. 3 originate from the matrix, while those labeled with small, filled circles are due to a loss of carboxylic acid groups, which is associated with the use of this matrix, but is apparently very minor. Accordingly, no signals originating from the peeling reactions were observable. Moreover, the intensity of the major signals observed using the described procedure were considerably stronger than those observed using hydrazinolysis (data not shown)

Microscale Release of Glycans. In initial experiments, the amounts of glycoproteins were used at the milligram level. This is high, considering the more typical amounts at which various glycoproteins may be encountered in challenging biological samples. Therefore, the procedure had to be scaled down to become more universally applicable. Using bovine fetuin quantities as low as 20 μg, the mass spectral ratios were successfully reproduced in comparison to FIG. 3 (data not shown).

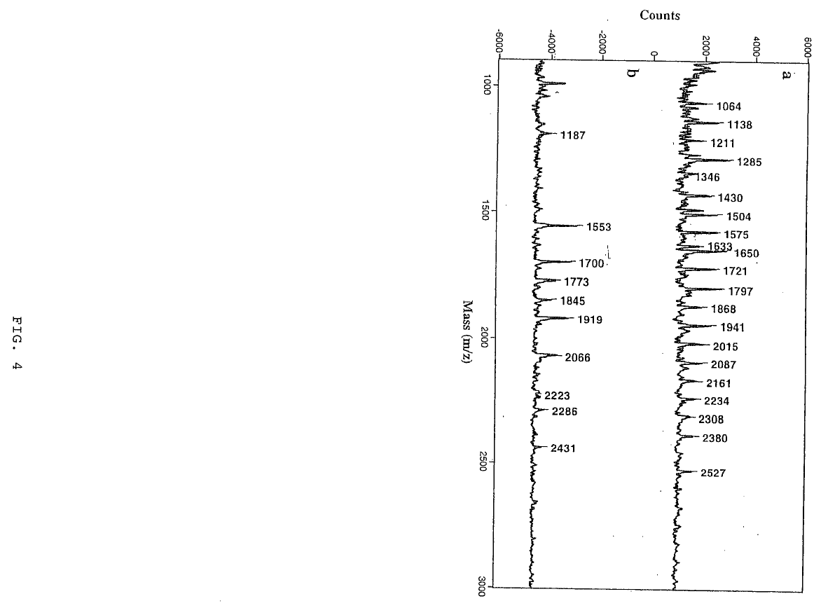

Another example of a more challenging glycoprotein (BSSL) is shown in FIG. 4. In total, a 10 μg amount of BSSL was processed to yield the spectra of glycans shown in this figure. It should be noted that the spectral recordings for both the positive and the negative ions were obtained from the spot corresponding to half of the amount of digested glycoprotein. BSSL is a relatively large glycoprotein consisting of 722 amino acid residues, with one N-glycosylation site toward the N-terminus, and numerous O-glycosylation sites nearer to the C-terminus. As shown in FIG. 4(a vs b), both sialylated (negative ions) and neutral (positive ions) structures were removed using the ammonia-based β-elimination.

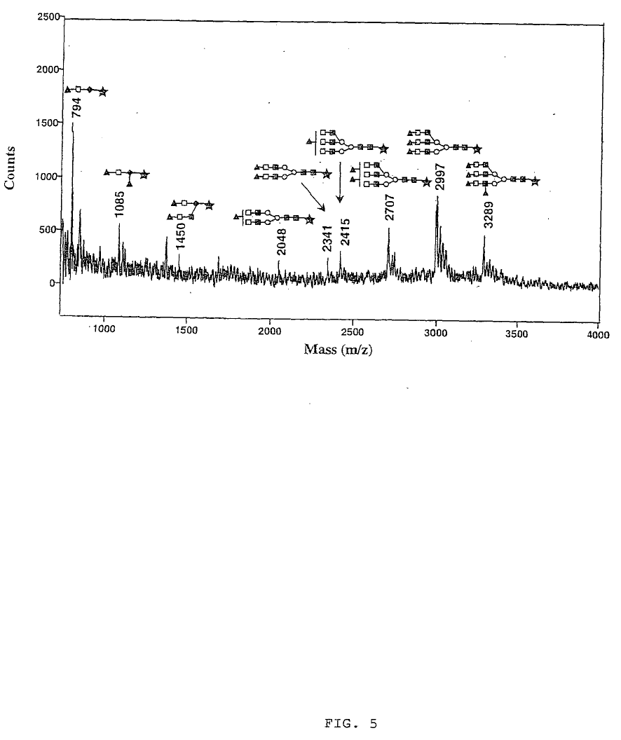

Derivatization of Reducing Glycans with 2-Aminobenzamide. The data presented in FIG. 2 provide sufficiently accurate mass determination of the previously known oligosaccharide composition of bovine fetuin to validate the ammonia-based β-elimination procedure although the difference between the reduced and nonreduced forms is only two mass units. Further supporting evidence can, however, be produced through the reactivity of their reducing end. The reaction with 2-aminobenzamide, via reductive amination provides this proof unequivocally (FIG. 5). Clearly, FIGS. 3 and 5 now feature the same bovine fetuin glycans. However, the spectral peaks in FIG. 5 are mass shifted by 120 m/z values due to incorporation of the benzamide moiety, indicating that the labeling has been successful. This could not have been accomplished with alditols. Moreover, the absence of signals due to unlabeled glycans suggests that conversion of glycosylamines to reducing glycans was complete prior to the use of a labeling reaction. The mass-shifting strategy will become generally useful in studying the profiles of O-linked glycans from the glycoproteins of unknown structures, as O-linked structures often feature short oligosaccharide chains.

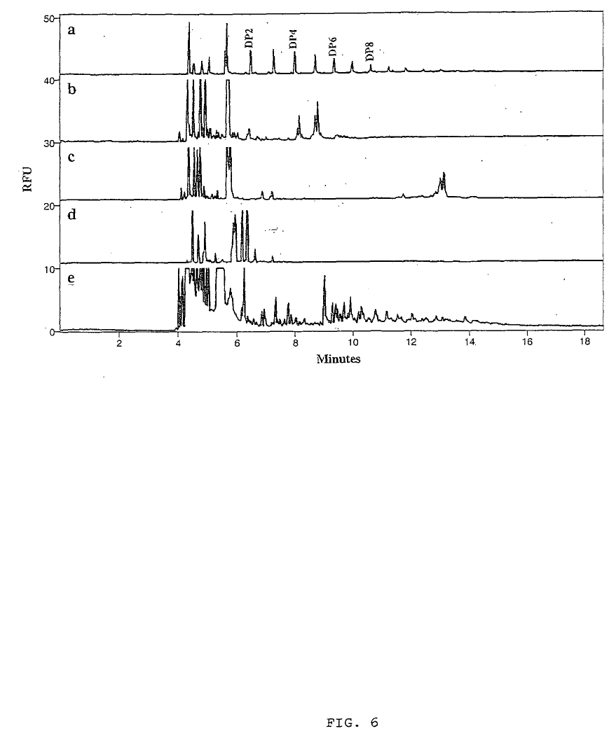

Capillary Electrophoresis of the APTS-Labeled Glycans. The practical utility of the described ammonia-based β-elimination protocol is further demonstrated in employing CE-LIF for mapping the mixtures of O-linked and N-linked oligosaccharides cleaved from microgram and submicrogram quantities of glycoproteins (FIG. 6). CE-LIF is an excellent means for a rapid profiling of oligosaccharide mixtures because of its high separation efficiency and detection sensitivity. However, this methodology has been thus far limited to N-linked oligosaccharides, since reductive animation was necessary prior to detection and the intact reducing O-linked glycans were rarely obtained. Through the present ammonia-based β-elimination protocol, both N-linked and O-linked oligosaccharides are simultaneously released from glycoproteins with intact reducing end, to be amenable to a labeling with any sensitive aromatic fluorophores that are commonly used in CE-LIF analysis. As examples, the electropherograms of the APTS-labeled glycans cleaved from different glycoproteins are shown in FIG. 6. FIG. 6a represents the dextran ladder used as the electromigration standard and was subjected to the ammonia-based β-elimination procedure prior to labeling with APTS.

FIG. 6a illustrates that interferences generated by any side reactions due to the ammonia-based P-elimination environment and APTS labeling are minimal. The difference in migration of certain components of fetuin and asialofetuin (FIG. 6b vs 6c) is consistent with the absence of negatively charged sialic acid residues in the latter. The glycans released from mucin (FIG. 6d) migrate relatively fast (<8 min), which is consistent with the previously established presence of O-linked glycans. The BSSL total oligosaccharide profile is apparently the most heterogeneous (FIG. 6e). Apparently, the ammonia-based β-elimination procedure releases both O-linked and N-linked glycan types from a glycoprotein.

The reproducibility of the ammonia-based β-elimination procedure was addressed by CE-LIF mapping of O-linked and N-linked oligosaccharides cleaved from glycoproteins. Using fetuin as the model, the glycans were cleaved from the glycoprotein and labeled with APTS prior to CE-LIF analysis. Samples prepared from different batches and at different times illustrated the high reproducibility of the protocol as suggested by the relative intensities of the peaks observed in the electropherograms (data not show).

The present ammonia-based β-elimination procedure provides a viable alternative to hydrazinolysis and the Carlson β-elimination methods. Its simplicity, the apparent lack of peeling reactions and deacetylation byproducts, combined with its effectiveness at microscale, are several advantages over the currently used methods. The described methodology is clearly superior to the Carlson β-elimination because it is capable of preserving the glycans with intact reducing terminals to be available for labeling. Moreover, the classical β-elimination has never been applicable to work at the low sample levels shown in this investigation. As demonstrated here, the method is also superior to hydrazinolysis because of its simplicity (one-pot procedure) and the absence of deacetylation byproducts. Minimum sample handling and easy removal of reactants make it possible to cleave effectively the oligosaccharide chains from only low-microgram quantities of glycoproteins.

The reported procedure is particularly effective in conjunction with the highly sensitive techniques of mass spectrometry and capillary separation methodologies. This methodology brings the analysis of O-glycans at par with the use of N-glycanases and sensitive measurements on N-glycans developed during the recent years. Conversion of O-linked oligosaccharides to the glycans with reducing ends allows their subsequent labeling with a fluorophore or any other structural entity that may facilitate better separation or detection capabilities.

### EXAMPLES

Materials and Chemicals. The human milk bile salt-stimulated lipase (from a pool of breast milk of over 100 individuals), which was prepared according to the procedure reported by Blackberg et al., was received from Astra-Zeneca (Molndal, Sweden). Bovine fetuin, bovine asialofetuin, and bovine submaxillary mucine type IS were purchased from Sigma Chemical Co. (St. Louis, Mo.). The fluorescence labeling reagent APTS was received from Molecular Probes, Inc. (Eugene, Oreg.). The other fluorophore (2-aminobenzamide) and all common chemicals were received from Aldrich (Milwaukee, Wis.).

Ammonia-Based β-Elimination. Typically, 1-mg amounts of a glycoprotein were dissolved in 1 mL of 28% aqueous ammonium hydroxide solution, which was saturated with ammonium carbonate at room temperature, in a 1.5-mL Eppendorf microtube. An additional 100 mg of ammonium carbonate solid was added to the reaction mixture. The mixture was subsequently incubated at 60° C. for 40 h. Ammonium hydroxide and ammonium carbonate were removed by repeated evaporation of water using Centra-Vap (Labconco Corp., Kansas City, Mo.) until no salts were noticeable in the microtube. Next, 10 μL of 0.5 M boric acid was added, and the mixture was incubated at 37° C. for 30 min. The tube was dried, and boric acid was removed by evaporation under a stream of nitrogen with several additions of methanol. Finally, the reaction mixture was dissolved in a 50-μL volume of water and centrifuged. The supernatant was used for MALDI-MS measurements and derivatization prior to CE-LIF analyses while the insoluble peptides were discarded. In the case of cleavage from 10 μg of sample (BSSL), the amounts of added reagents were proportionally reduced.

2-Aminobenzamide Labeling. Labeling of oligosaccharides with 2-aminobenzamide was performed according to a published procedure. Briefly, 0.35 M 2-aminobenzamide and 1 M sodium cyanoborohydride solutions were separately prepared in the mixture of acetic acid/dimethyl sulfoxide (3:7). A 5-μL aliquot of each solution was added to a dried oligosaccharide sample, while the mixture was stirred and incubated in the dark at 60° C. for 4 h. The reaction mixture was dried under vacuum and dialyzed overnight against high-purity water using a MWCO-500 dialysis membrane (Spectrum, Houston, Tex.). It was then subjected to MS analysis.

APTS Labeling. The derivatization method followed a published procedure. A 5-μL aliquot of 40 mM APTS solution was added to an oligosaccharide sample, followed by the addition of 0.5 μL of concentrated acetic acid and 0.5 μL of 1.0 M sodium cyanoborohydride. After incubation at 60° C. for 4 h, the mixture was diluted by a 200-μL volume of water and stored at −20° C. until the CE-LIF analysis was performed. Further 10-1000-fold dilutions were needed before sample introduction.

MALDI-Time-of-Flight Mass Spectrometry. Mass spectra were acquired on a Voyager-DE RP Biospectrometry Workstation instrument (Applied Biosystems, Framingham, Mass.) equipped with a pulsed nitrogen laser (337 nm). MALDI spectra were acquired at 25 and 18 kV accelerating voltage in the positive- and negative-ion modes, respectively, while the low-mass gate was used to discard the ions with m/z values of less than 400. All acquired spectra were smoothed by applying a 19-point Savltzky-Golay smoothing routine. The matrix used was 10 mM 2,5-dihydroxybenzoic acid prepared in a 1:1 mixture of ethanol and a 25 mM spermine aqueous solution. Prior to MS analysis, samples were briefly desalted. The sampling spots were dried under vacuum. They could be analyzed in both positive- and negative-ion mode.

Capillary Electrophoresis. The instrument for capillary electrophoresis was assembled in-house from the commercially available components, as described earlier. It utilized a high-voltage power supply (0-40 kV) from Speilman High Voltage Electronics (Plainview, N.J.). A 488-nm argon ion laser, from Omnichrome (Chino, Calif.) was used as the light source. Fluorescence emission at 514 nm was collected through a microscope lens and monitored using R928 photomultiplier tube (Hamamatsu Photonics K.K., Shizuoka Prefecture, Japan). The signal was amplified with a lock-in amplifier (EG&G Princeton Applied Research, Princeton, N.J.). The separation was conducted in a fused-silica capillary (50 cm effective length, 60 cm total length, and 50 μm i.d.) coated with linear polyacrylamide.

1. A method for preparing stable oligosaccharides from a glycoprotein having linked oligosaccharides, said method comprising the steps o
contacting said glycoprotein with an aqueous solution of ammonium hydroxide and ammonium carbonate for a period of time sufficient to cleave linked oligosaccharides from the glycoprotein to form oligosaccharide products and a protein by-product;
separating the oligosaccharide products from the ammonium hydroxide and the ammonium carbonate; and
separating at least a portion of the oligosaccharide products from the protein by-product.

2. The method of claim 1 further comprising the step of contacting the oligosaccharide products with an aqueous acid.

3. The method of claim 2 wherein the acid is boric acid.

4. The method of claim 2 further comprising the step of separating the oligosaccharide products from the acid.

5. The method of claim 3 further comprising the step of separating the oligosaccharide products from the acid.

6. The method of claim 1 further comprising the step of separating at least one oligosaccharide products from other oligosaccharide products.

7. The method of claim 2 further comprising the step of separating at least one oligosaccharide products from other oligosaccharide products.

8. The method of claim 3 further comprising the step of separating at least one oligosaccharide products from other oligosaccharide products.

9. The method of claim 4 further comprising the step of separating at least one oligosaccharide products from other oligosaccharide products.

10. The method of claim 5 further comprising the step of separating at least one oligosaccharide products from other oligosaccharide products.

11. The method of claim 1 further comprising the step of reacting the separated oligosaccharide products with a labeling agent to form a mixture of oligosaccharide derivatives having a common covalently bound label.

12. The method of claim 2 further comprising the step of reacting the separated oligosaccharide products with a labeling agent to form a mixture of oligosaccharide derivatives having a common covalently bound label.

13. The method of claim 3 further comprising the step of reacting the separated oligosaccharide products oligosaccharides with a labeling agent to form a mixture of oligosaccharide derivatives having a common covalently bound label.

14. The method of claim 4 further comprising the step of reacting the separated oligosaccharide products with a labeling agent to form a mixture of oligosaccharide derivatives having a common covalently bound label.

15. The method of claim 5 further comprising the step of reacting the separated oligosaccharide products oligosaccharides with a labeling agent to form a mixture of oligosaccharide derivatives having a common covalently bound label.

16. The method of claim 11 further comprising the step of separating at least one labeled oligosaccharide derivative from other labeled oligosaccharide derivatives.

17. The method of claim 12 further comprising the step of separating at least one labeled oligosaccharide derivative from other labeled oligosaccharide derivatives.

18. The method of claim 13 further comprising the step of separating at least one labeled oligosaccharide derivative from other labeled oligosaccharide derivatives.

19. The method of claim 14 further comprising the step of separating at least one labeled oligosaccharide derivative from other labeled oligosaccharide derivatives.

20. The method of claim 15 further comprising the step of separating at least one labeled oligosaccharide derivative from other labeled oligosaccharide derivatives.

21. A method for preparing stable reducing oligosaccharides by cleavage of a glycoprotein having linked oligosaccharides, said method comprising the steps of
contacting the glycoprotein with an aqueous solution of ammonium hydroxide and ammonium carbonate to cleave the linked glycans and from intermediate product oligosaccharide products and cleaved protein by-products;
separating the intermediate products from the aqueous ammonium hydroxide and ammonium carbonate;
reacting the intermediate oligosaccharide products with aqueous boric acid to form reducing oligosaccharide products; and
separating at least a portion of the reducing oligosaccharide products from the aqueous boric acid and the protein by-products.

22. The method of claim 21 wherein the step of separating the aqueous ammonium hydroxide and ammonium carbonate from the intermediate oligosaccharide products is carried out by evaporation of aqueous solutions of the products and those reagents.

23. The method of claim 21 wherein the reducing oligosaccharide products are separated from the boric acid by evaporation of methanol solutions of the product mixture.

24. The method of claim 21 further comprising the step of separating at least one reducing oligosaccharide product from the other reducing oligosaccharide products.
